(19) United States
(12) Patent Application Publication
(10) Pub. No.: US 2023/0035590 A1
(43) Pub. Date: Feb. 2, 2023

(54) LIGHT-GUIDING STRUCTURE, ENDOSCOPE TIP, AND METHOD FOR MANUFACTURING A LIGHT-GUIDING STRUCTURE

(71) Applicant: ALTEK BIOTECHNOLOGY CORPORATION, Hsinchu City (TW)

(72) Inventors: Hsi-Hsin Loo, Hsinchu City (TW); Liang-Yi Li, Hsinchu City (TW)

(73) Assignee: ALTEK BIOTECHNOLOGY CORPORATION, Hsinchu City (TW)

(21) Appl. No.: 17/389,326

(22) Filed: Jul. 29, 2021

(51) Int. Cl. G02B 23/24

(52) U.S. Cl. CPC G02B 23/2469; G02B 23/2484

(57) ABSTRACT

A light-guiding structure according to the invention is used in an endoscope for guiding light along a lengthwise direction and includes a first portion and a second portion. The first portion extends in a single cross section along the lengthwise direction. The second portion extends in a varying cross section along the lengthwise direction and is connected to an end of the first portion in the lengthwise direction. An endoscope tip includes a circuit board, an image-capturing component, a light-emitting component, and the light-guiding structure. The light-guiding structure can fit the contours of the circuit board and the image-capturing component to increase space usage for obtaining more cross-sectional area. For the production of the light-guiding structure, the second portion is formed by shaping a portion directly extending form the end of the first portion or by an additional material directly bonded to the end of the first portion by molding.

### BACKGROUND OF THE INVENTION

### 1. Field of the Invention

The present invention relates to a light-guiding structure, and more particularly to a light-guiding structure used in an endoscope and a method for manufacturing the light-guiding structure.

### 2. Description of the Prior Art

A general endoscope mainly includes a control handle and a long insertion tube connected to the control handle. A plurality of channels are formed in the insertion tube for passage of required objects, such as electrical wires, optical fibers, bending control cables, instruments, etc. An end of the insertion tube is usually equipped with an image-capturing device. The light required for capturing images can be generated by an external light source and reach the end through optical fibers to illuminate the object to be photographed. A user can hold and manipulate the control handle to bend the insertion tube, capture images, operate instruments, and so on. In general, in order to provide sufficient intensity of light when capturing images, the optical fiber (or a bundle of optical fibers) needs to have a certain outer diameter. Furthermore, considering the attenuation of light transmission, as the length of the insertion tube increases, the diameter of the optical fiber (or a bundle of optical fibers) also needs to be increased. It is difficult to reduce the outer diameter of a longer insertion tube, which limits the scope of application. In addition, longer fibers are more expensive to manufacture, and the extension connection between optical fibers will increase attenuation.

### SUMMARY OF THE INVENTION

An objective of the invention is to provide a light-guiding structure, which can replace the traditional optical fiber and be used in an endoscope, so that the diameter of the insertion tube of the endoscope can be reduced.

A light-guiding structure according to the invention is used in an endoscope for guiding light along a lengthwise direction and includes a first portion and a second portion. The first portion extends in a single cross section along the lengthwise direction. The second portion extends in a varying cross section along the lengthwise direction and is connected to an end of the first portion in the lengthwise direction. Therein, the second portion is formed by shaping a portion directly extending form the end of the first portion or by an additional material directly bonded to the end of the first portion by molding. Thereby, the second portion can be formed in accordance with the structure configuration of the insertion tube end of the endoscope equipped with the light-guiding structure. A light source can be disposed relatively close to the insertion tube end, so the outer diameter of the insertion tube of the endoscope can be reduced. Light emitted by the light source is guided by the light-guiding structure to smoothly emit from the insertion tube end. Furthermore, the length of the travelling path of the light through the light-guiding structure is shorter than that through the optical fiber in a conventional endoscope, so the attenuation of light transmission is lower and the power of the light source is smaller.

Another objective of the invention is to provide an endoscope tip, equipped with a light-guiding structure. The light-guiding structure can replace the traditional optical fiber, so that the diameter of the insertion tube of an endoscope equipped with the endoscope tip can be reduced.

An endoscope tip according to the invention includes a circuit board, an image-capturing component, a light-emitting component, and the aforementioned light-guiding structure. The image-capturing component and the light-emitting component are disposed on the circuit board. The light-guiding structure is disposed above the circuit board for guiding light emitted by the light-emitting component along a lengthwise direction. Thereby, the second portion can be formed in accordance with the structure configuration of the endoscope tip (including the arrangement of the circuit board, the image-capturing component, and other components or channels e.g. for instruments). The outer diameter of the insertion tube of an endoscope equipped with the endoscope tip can be reduced. The light emitted by the light-emitting component is guided by the light-guiding structure to smoothly emit from the endoscope tip. Furthermore, the length of the travelling path of the light through the light-guiding structure is shorter than that through the optical fiber in a conventional endoscope, so the attenuation of light transmission is lower and the power of the light-emitting component is smaller.

Another objective of the invention is to provide a method for manufacturing a light-guiding structure used in an endoscope. The method uses secondary molding to make the light-guiding structure, which can increase the structural adaptability of the light-guiding structure.

According to an embodiment of the invention, a method for manufacturing a light-guiding structure used in an endoscope includes the following steps: (a) providing a light-transmissive structure, the light-transmissive structure extending in a single cross section along a lengthwise direction and having a first end and a second end opposite to the first end in the lengthwise direction; and (b) shaping a portion of the light-transmissive structure with the second end so that the shaped portion extends in a varying cross section along the lengthwise direction, so as to complete the light-guiding structure. Thereby, the second portion can be formed as required without being restricted by the structure of the first portion, so that the overall structural adaptability of the light-guiding structure is increased. In practice, the light-guiding structure can replace the traditional optical fiber and be used in an endoscope or an endoscope tip, so that the diameter of the insertion tube of the endoscope or an endoscope equipped with the endoscope tip can be reduced.

According to another embodiment of the invention, a method for manufacturing a light-guiding structure used in an endoscope includes the following steps: (a) providing a light-transmissive structure, the light-transmissive structure extending in a single cross section along a lengthwise direction and having a first end and a second end opposite to the first end in the lengthwise direction; (b) providing a mold with a cavity; (c) disposing the light-transmissive structure in the mold so that the second end is exposed in the cavity; (d) filling the cavity with a material into the cavity; and (e) solidifying the material in the cavity so that the solidified material is directly bonded to the second end and extends in a varying cross section along the lengthwise direction, so as to complete the light-guiding structure. Thereby, the second portion can be formed as required (e.g. by designing the size of the cavity) without being restricted by the structure of the first portion, so that the overall structural adaptability of the light-guiding structure is increased. In practice, the light-guiding structure can replace the traditional optical fiber and be used in an endoscope or an endoscope tip, so that the diameter of the insertion tube of the endoscope or an endoscope equipped with the endoscope tip can be reduced.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating an endoscope according to an embodiment.

FIG. 2 is a schematic diagram illustrating an endoscope tip of the endoscope in FIG. 1.

FIG. 3 is a partially exploded view of the endoscope tip in FIG. 2.

FIG. 4 is a schematic diagram illustrating a light-guiding structure of the endoscope tip in FIG. 3 from another view point.

FIG. 5 is a sectional view of the light-guiding structure in FIG. 3 along its lengthwise direction; therein, other surrounding structures are shown in dashed lines.

FIG. 6 is a cross-section of the light-guiding structure along the line X-X in FIG. 4.

FIG. 7 is a cross-section of the light-guiding structure along the line Y-Y in FIG. 4.

FIG. 8 is a sectional view of the light-guiding structure without a third portion, the circuit board, and the light-emitting component of the endoscope tip according to an instance.

FIG. 9 is a sectional view of the light-guiding structure without a third portion, the circuit board, and the light-emitting component of the endoscope tip according to another instance.

FIG. 10 is a flowchart of a method for manufacturing a light-guiding structure.

FIG. 11 is a schematic diagram illustrating a light-transmissive structure and a mold for shaping the light-transmissive structure.

FIG. 12 is a schematic diagram illustrating the light-transmissive structure in FIG. 11 that is shaped to have a second portion.

FIG. 13 is a schematic diagram illustrating the light-transmissive structure in FIG. 12 that is further shaped to have a third portion.

FIG. 14 is a schematic diagram illustrating the light-transmissive structure is shaped to have a light-incident surface oblique to the lengthwise direction according to an instance.

FIG. 15 is a flowchart of another method for manufacturing a light-guiding structure.

FIG. 16 is a schematic diagram illustrating a light-transmissive structure and a mold for forming a second portion directly connecting with the light-transmissive structure.

### DETAILED DESCRIPTION

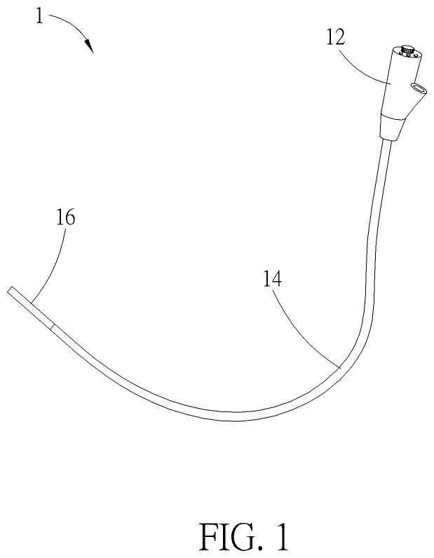

Please refer to FIG. 1 to FIG. 5. An endoscope 1 according to an embodiment includes a control handle 12 and an insertion tube 14. The insertion tube 14 has a plurality of channels for passage of required objects, such as electrical wires, bending control cables, instruments, water or other liquids, etc., which are not shown in the figures for drawing simplification. The insertion tube 14 has an endoscope tip 16 at one end and is connected to the control handle 12 through the other end. A user can hold and manipulate the control handle 12 to control the direction of the endoscope tip 16 and operate instruments, and so on.

In the embodiment, the endoscope tip 16 includes a tip housing 160, a circuit board 162, an image-capturing component 164, a light-emitting component 166, and a light-guiding structure 168. The tip housing 160 (shown by a thin tube for drawing simplification) accommodates the circuit board 162, the image-capturing component 164, the light-emitting component 166, and the light-guiding structure 168. The circuit board 162 is electrically connected to the control handle 12 (e.g. by electrical wires passing through the channels), so that the control handle 12 can provide power to the circuit board 162 and control the operation of the circuit board 162. In practice, the channels also extend to the tip housing 160, so that the instruments can protrude from the endoscope tip 16; for example, the tip housing 160 forms part of the channel directly or in coordination with other insertion parts. The image-capturing component 164 is disposed on the circuit board 162 and exposed at one end of the tip housing 160 (or the tip end of the endoscope tip 16), so that the image-capturing component 164 can be controlled to capture images in front of the endoscope tip 16. The light-emitting component 166 is disposed on the circuit board 162 to provide the light required by the image-capturing component 164 to capture images. The light-guiding structure 168 is disposed above the circuit board 162 and between the image-capturing component 164 and the light-emitting component 166 for guiding the light emitted by the light-emitting component 166 along a lengthwise direction 188a (indicated by a double-head arrow in the figures) to illuminate the front of the endoscope tip 16.

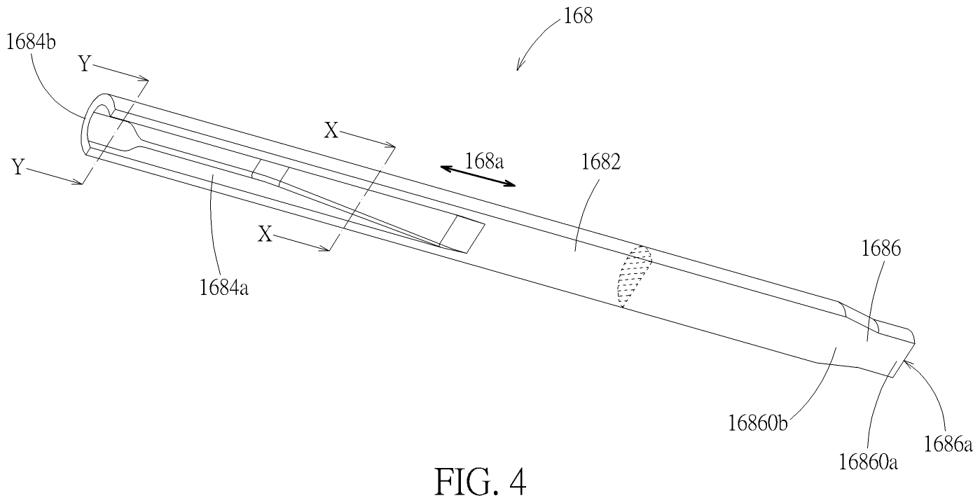

In the embodiment, the light-guiding structure 168 includes a first portion 1682, a second portion 1684, and a third portion 1686. The first portion 1682 extends in a single cross section along the lengthwise direction 168a. The cross section may be designed to match its surrounding structures, such as the circuit board 162 and/or the tip housing 160, so that the first portion 1682 will not structurally interfere with them. In the embodiment, the cross section (illustrated by a dashed area filled with hatching in FIG. 4) of the first portion 1682 is a circular segment with a central angle greater than 180 degrees and fits the contours of the circuit board 162. However, it is not limited thereto.

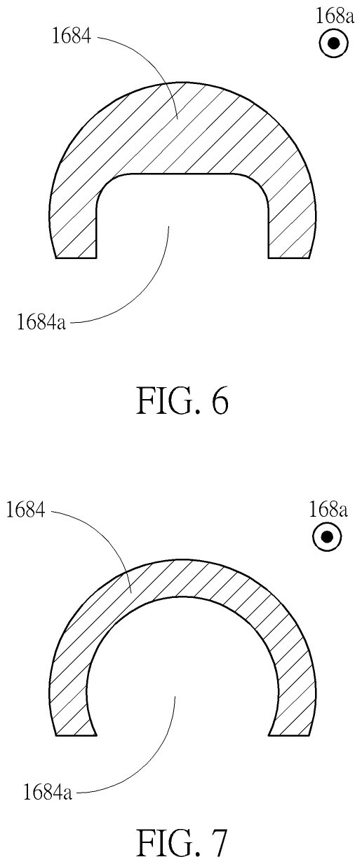

The second portion 1684 is connected to an end 1682a of the first portion 1682 in the lengthwise direction 168a; in other words, the first portion 1682 is close to the light-emitting component 166 relative to the second portion 1684. The second portion 1684 extends in a varying cross section along the lengthwise direction 168a. Similarly, the cross section of the second portion 1684 may be designed to match its surrounding structures, such as the image-capturing component 164 and/or the tip housing 160, so that the second portion 1684 will not structurally interfere with them. In the embodiment, as shown by FIG. 6, the outer side the cross section of the second portion 1684 relatively close to the first portion is an arc shape (with a central angle greater than 180 degrees); the inner side is n-shaped for avoiding structural interference with the upwardly extending circuit board 162. As shown by FIG. 7, the cross section of the second portion 1684 relatively away from the first portion is an arc-shaped thin shell for fitting the contours of the tip housing 160 and the image-capturing component 164. On the other hand, the second portion 1684 has a component accommodating space 1684a, and the image-capturing component 164 is at least partially accommodated in the component accommodating space 1684a. In practice, the varying cross section of the second portion 1684 is not limited to the case where the cross section continuously changes. The change trend of the cross section of the second part 1684 along the length direction 168a depends in principle on its surrounding structures. In addition, for example, the second portion 1684 may be formed by shaping a portion directly extending form the end 1682a of the first portion 1682 (i.e. the first and second portions are formed as a single part of the same material) or by an additional material directly bonded to the end 1682a of the first portion 1682 by molding. Thereby, the second portion 1684 can be formed as required (e.g. by designing the mold for the molding) without being restricted by the structure of the first portion 1682, so that the overall structural adaptability of the light-guiding structure 168 is increased.

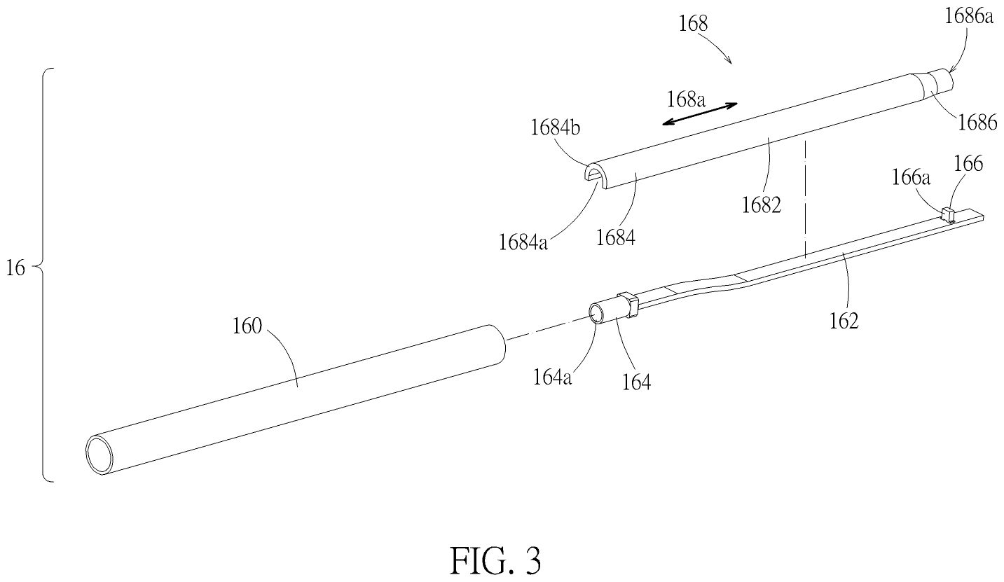

Please refer back to FIG. 3 to FIG. 5. The third portion 1686 is connected to another end 1682b of the first portion 1682 opposite to the second portion 1684 and extends in a varying cross section along the lengthwise direction 168a. The third portion 1686 is between the first portion 1682 and the light-emitting component 166. Thereby, the light emitted by the light-emitting component 166 enters the light-guiding structure 168 from the third portion 1686, passes through the first portion 1682, and emits out the light-guiding structure 168 from the second portion 1684. The third portion 1686 is slightly tapered. The cross section of the third portion 1686 gradually changes; therein, the cross section of the third portion 1686 close to the free end 16860a is smaller than the cross section of the third portion 1686 close to the connection end 16860b (to the first portion 1682). It helps the light be guided through the light guiding structure 168 and benefit the uniformity of output light distribution from the light-guiding structure 168.

Therefore, in the embodiment, the light guide structure 168 utilizes the available space in the tip housing 160 as much as possible. Compared with the light guide effect limited by the fiber diameter in the prior art, the light guide structure 168 helps to increase the light guide cross-sectional area and improve the light guide effect. Furthermore, the light-emitting component 166 (i.e. light source) is disposed in the endoscope tip 16 (i.e. relatively close to the object to be inspected), so the attenuation of light transmission is lower and the power for the light-emitting component 166 to emit the light is smaller. Therein, because the light-emitting component 166 is still at a distance from the object to be inspected, the heat generated by the light-emitting component 166 during operation will not affect the object in principle.

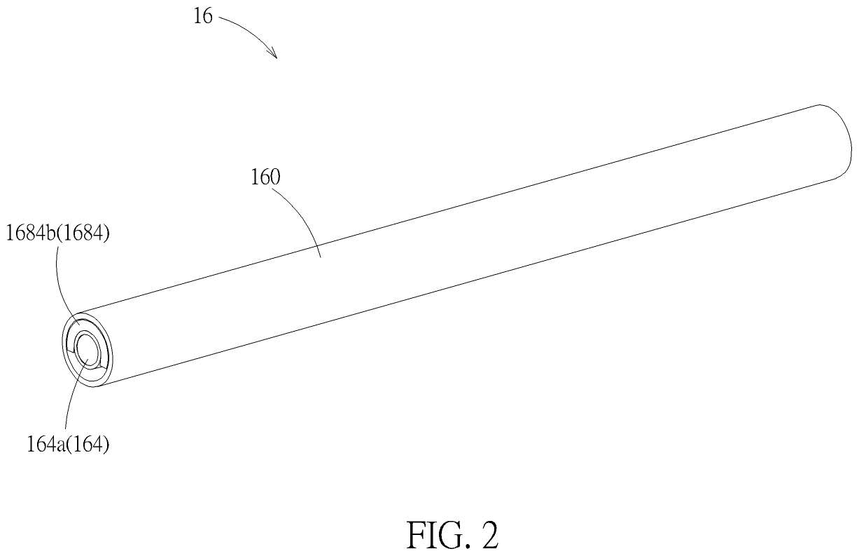

In addition, in the embodiment, as shown by FIG. 2 to FIG. 5, the second portion 1684 an annular segment light-emitting surface 1684b at a side of the component accommodating space 1684a adjacent to a lens 164a of the image-capturing component 164. Compared with the illumination formed by the point light source formed by the ends of the optical fibers in the prior art, the annular segment light-emitting surface 1684b helps to uniformly illuminate the object in front of the endoscope tip 16; that is, it helps to obtain qualified images more easily.

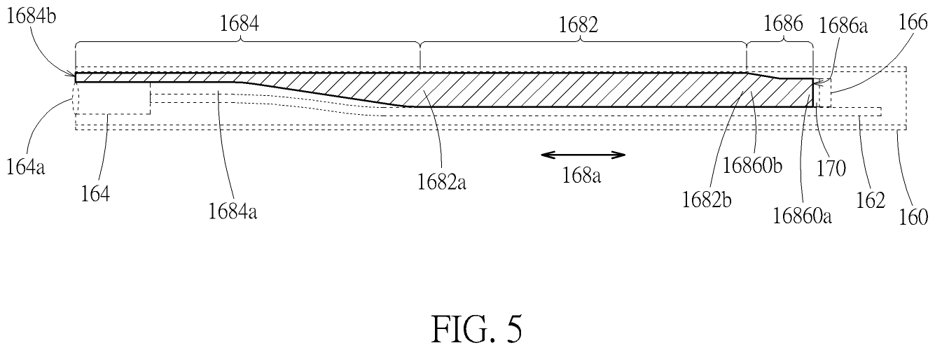

In the embodiment, the free end 16860a of the third portion 1686 is used as a light-incident surface 1686a, through which the light emitted by the light-emitting component 166 enters the light-guiding structure 168. The light-incident surface 1686a is flat. The light-emitting component 166 has a light-emitting surface 166a parallel to the light-incident surface 1686a. In practice, the light-incident surface 1686a and the light-emitting surface 166a may be bonded with an optically clear adhesive 170 (indicated in FIG. 5). In addition, if the light-guiding structure 168 may be provided without the third portion 1686, the free end of the first portion 1682 is used as a light-incident surface for receiving the light emitted by the light-emitting component 166.

For increasing the uniformity of output light distribution from the light-guiding structure 168, it is practicable to design the light-incident surface of the light-guiding structure 168 and the relative disposition of the light-incident surface of the light-guiding structure 168 and the light-emitting surface (or the nominal light-emitting direction) of the light-emitting component 166. For simplification of illustration, the light-guiding structure 168 will be simplified to be without the third portion 1686 in the following description and relevant figures. As shown by FIG. 8 (without hatch lines for drawing simplification), the free end 1682a′ of the first portion 1682 is used as a flat light-incident surface 1682c. The normal direction 1682d of the flat light-incident surface 1682c and the lengthwise direction 168a form an acute angle 168b. The light-emitting surface 166a and the flat light-incident surface 1682c are parallel. Thereby, more light enters the light-guiding structure 168 (from the flat light-incident surface 1682c) at a larger angle with the lengthwise direction 168a, so that more light emits out the light-guiding structure 168 at a larger refraction angle, which helps to increase the uniformity of the output light distribution from the light-guiding structure 168 and also helps to obtain qualified images more easily (for example, to avoid overexposure of the images). In practice, the acute angle 168b may be greater than 0 degree and not greater than 15 degrees. Furthermore, if the light-emitting surface 166a is close to the light-incident surface 1682c enough (e.g. about 0.10 mm relative to 0.15 mm) and an optically clear adhesive is filled between them, the uniformity of the output light distribution will be significantly improved as the acute angle 168b is 15 degrees.

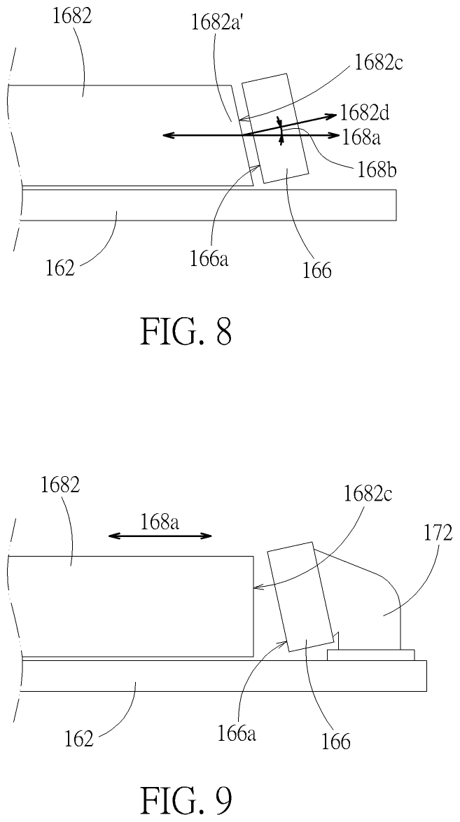

In another instance (as shown by FIG. 9), similar to FIG. 8, but the flat light-incident surface 1682c is perpendicular to the lengthwise direction 168a. The direction of the light-emitting component 166 is adjustable, which may be achieved by adjustably disposing the light-emitting component 166 on the circuit board 162 through an adjustable mechanism 172 (e.g. including rotation and one-dimensional or two-dimensional movement). The adjustable mechanism 172 may be set before the factory or by the user afterwards (e.g. through the circuit board 162 by manipulating on the control handle). Similarly, more light enters the light-guiding structure 168 (from the flat light-incident surface 1682c) at a larger angle with the lengthwise direction 168a, so that more light emits out the light-guiding structure 168 at a larger refraction angle, which helps to increase the uniformity of the output light distribution from the light-guiding structure 168 and also helps to obtain qualified images more easily. In practice, because the adjustable mechanism 172 can adjust the direction of the light-emitting component 166 relative to the flat light-incident surface 1682c, the flat light-incident surface 1682c may not be perpendicular to the lengthwise direction 168a, and may form other included angles with the lengthwise direction 168a according to requirements, which will not be described in detail. In addition, the above two ways for increase the uniformity of the output light distribution from the light-guiding structure 168 are also applicable to the light-incident surface 1686a (i.e. at the free end 16860a) of the third portion 1686, which will not be repeated in detail.

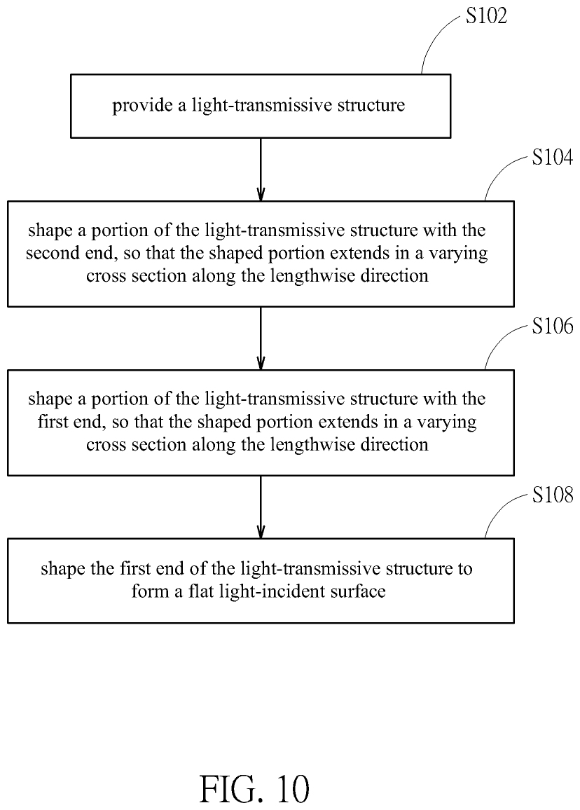

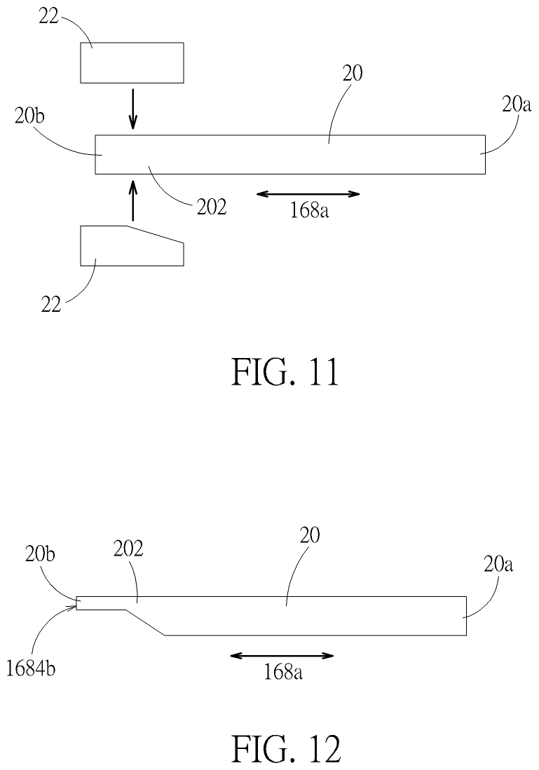

Please refer to FIG. 10. A method for manufacturing a light-guiding structure (e.g. the light-guiding structure 168) is to provide a light-transmissive structure 20, as shown by the step S102. For simplification of illustration, the reference numbers used by the light-guiding structure 168 will in principle continue to be used in the following description. Besides, for drawing simplification, the structures mentioned in the following description will be shown in sectional views without hatch lines. As shown by FIG. 11, the light-transmissive structure 20 (shown exaggeratedly in size) extends in a single cross section along a lengthwise direction 168a and has a first end 20a and a second end 20b opposite to the first end 20a in the lengthwise direction 168a. For description about the cross section of the light-transmissive structure 20, please refer to the relevant descriptions of the cross section of the first portion 1682 of the light-guiding structure 168, which will not be repeated in addition. In practice, the light-transmissive structure 20 may be formed but not limited by extrusion.

Afterwards, the method is to shape a portion 202 of the light-transmissive structure 20 with the second end 20b, so that the shaped portion extends in a varying cross section along the lengthwise direction 168a, as shown by the step 104. In an instance, as shown by FIG. 11 and FIG. 12, the step S104 includes using a mold 22 to shaping the portion 202 of the light-transmissive structure 20; therein, the shaped portion 202 (equivalent to the second portion 1684) is shown as FIG. 12. For increasing the material fluidity of the portion 202, the step S104 may include heating the mold 22 before the mold 22 shapes the portion 202 of the light-transmissive structure 20. In addition, for description about the cross section of the shaped portion 202, please refer to the relevant descriptions of the cross section of the second portion 1684 of the light-guiding structure 168, which will not be repeated in addition. Furthermore, in practice, the contours of the shaped portion 202 depend on the design of the mold 22. In the instance, the portion 202 is shaped to the second portion 1684. Therefore, in the step S104, the method is to use the mold 22 to shape the portion 202 of the light-transmissive structure 20 with the second end 20b so that the shaped portion 202 (equivalent to the second portion 1684) has a component accommodating space 1684a and an annular segment light-emitting surface 1684b at a side of the component accommodating space 1684a (also referring to FIG. 2 to FIG. 5). In practice, the shape of the shaped portion 202 and the size of the component accommodating space 1684a of the shaped portion 202 can be formed as required (e.g. by designing the mold 22) without being restricted by the structure of the first portion 1682, so that the overall structural adaptability of the light-guiding structure 168 is increased.

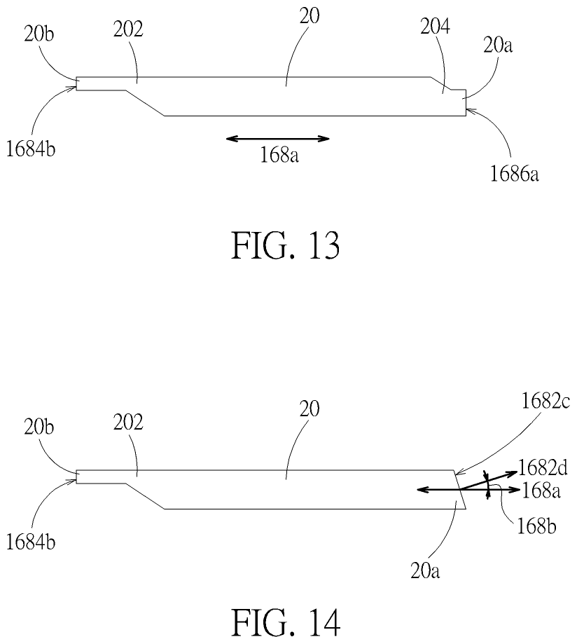

Afterwards, the method is to shape a portion 204 of the light-transmissive structure 20 with the first end 20a, so that the shaped portion 204 extends in a varying cross section along the lengthwise direction 168a, as shown by the step 106. As shown by FIG. 13, the shaped portion 204 is taken as the third portion 1686 (also referring to FIG. 3 to FIG. 5). For description about the cross section of the shaped portion 204, please refer to the relevant descriptions of the cross section of the third portion 1686 of the light-guiding structure 168, which will not be repeated in addition. Furthermore, in the instance, the free end (equivalent to the end 20a) of the shaped portion 204 (equivalent to third portion 1686) is used as a light-incident surface 1686a. Similarly, the shaping of the third portion 1686 also can be achieved but not limited by another mold. In addition, in FIG. 13, the light-transmissive structure 20 except for the second and third portions 1684 and 1686 is taken as the first portion 1682.

As described in the foregoing, for increasing the uniformity of output light distribution from the light-guiding structure 168, the light-incident surface of the light-guiding structure 168 may be shaped further. For simplification of illustration, the following is based on FIG. 12. As shown by FIG. 10 and FIG. 14, the method is to shape the first end 20a of the light-transmissive structure 20 to form a flat light-incident surface 1682c (also referring to FIG. 9), as shown by the step S108. Therein, a normal direction 1682d of the flat light-incident surface 1682c and the lengthwise direction 168a form an acute angle 1682d. For other descriptions of the flat light-incident surface 1682c, please refer to the relevant descriptions and figures in the foregoing, which will not be repeated in addition.

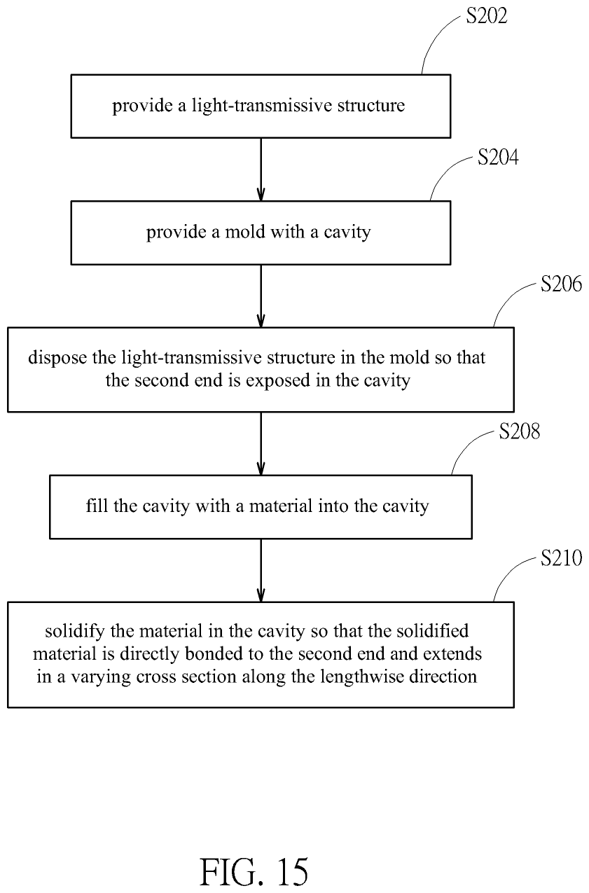

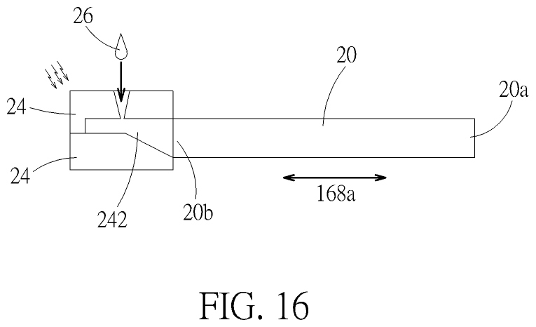

Please refer to FIG. 15. Another method for manufacturing a light-guiding structure (e.g. the light-guiding structure 168) is similar to the above method. A difference between them is the forming of the second portion 1684. For simplification of illustration, the following description will focus on the forming of the second portion 1684. For the descriptions of the forming of the other portions of the light-guiding structure 168 and variants thereof, please refer to the relevant descriptions and figures, which will not be repeated. As shown by FIG. 15, the method is to provide a light-transmissive structure 20, as shown by the step S202. As shown by FIG. 16 (or referring to FIG. 11), the light-transmissive structure 20 (shown exaggeratedly in size) extends in a single cross section along the lengthwise direction 168a and has the first end 20a and the second end 20b opposite to the first end 20a in the lengthwise direction 168a.

Afterwards, as shown FIG. 15 and FIG. 16, the method is to provide a mold 24 with a cavity 242, as shown by the step S204; the method is then to dispose the light-transmissive structure 20 in the mold 24 so that the second end 20b is exposed in the cavity 242, as shown by the step S206; the method is then to fill the cavity 242 with a material 26 into the cavity 242 (for example, but not limited to by injection), as shown by the step S208. Afterwards, the method is to solidify the material 26 in the cavity so that the solidified material (used as the second portion 1684) is directly bonded to the second end 20b and extends in a varying cross section along the lengthwise direction 168a, as shown by the step S210; the workpiece after de-molding is shown by FIG. 13 and will not be repeated in detail. In addition, according to the choice of the material 26, the material 26 may be solidified in different ways. For example, if the material 26 is photopolymer, in the step S210, the method is to curing the material 26 in the cavity 242 with a light (e.g. ultraviolet rays); therein, the mold 24 is made of ultraviolet transmissive material. Similarly, in practice, the shape of the second portion 1684 (i.e. the solidified material) and the size of the component accommodating space 1684a of the second portion 1684 can be formed as required (e.g. by designing the size of the cavity 242) without being restricted by the structure of the first portion 1682, so that the overall structural adaptability of the light-guiding structure 168 is increased.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A light-guiding structure used in an endoscope for guiding light along a lengthwise direction, the light-guiding structure comprising:
a first portion, extending in a single cross section along the lengthwise direction; and
a second portion, extending in a varying cross section along the lengthwise direction and connected to an end of the first portion in the lengthwise direction, wherein the second portion is formed by shaping a portion directly extending form the end of the first portion or by an additional material directly bonded to the end of the first portion by molding.

2. The light-guiding structure according to claim 1, wherein the second portion has a component accommodating space.

3. The light-guiding structure according to claim 2, wherein the second portion has an annular segment light-emitting surface at a side of the component accommodating space.

4. The light-guiding structure according to claim 1, further comprising a third portion, wherein the third portion is connected to another end of the first portion opposite to the second portion and extends in a varying cross section along the lengthwise direction.

5. The light-guiding structure according to claim 1, wherein the light-guiding structure has a flat light-incident surface, and a normal direction of the flat light-incident surface and the lengthwise direction form an acute angle.

6. The light-guiding structure according to claim 1, wherein the first and second portions are formed as a single part of the same material.

7. An endoscope tip, comprising:
a circuit board;
an image-capturing component, disposed on the circuit board;
a light-emitting component, disposed on the circuit board; and
a light-guiding structure, disposed above the circuit board for guiding light emitted by the light-emitting component along a lengthwise direction, the light-guiding structure comprising:
a first portion, close to the light-emitting component and extending in a single cross section along the lengthwise direction; and
a second portion, extending in a varying cross section along the lengthwise direction and connected to an end of the first portion in the lengthwise direction, wherein the second portion is formed by shaping a portion directly extending form the end of the first portion or by an additional material directly bonded to the end of the first portion by molding.

8. The endoscope tip according to claim 7, wherein the second portion has a component accommodating space, and the image-capturing component is at least partially accommodated in the component accommodating space.

9. The endoscope tip according to claim 8, wherein the second portion has an annular segment light-emitting surface at a side of the component accommodating space adjacent to a lens of the image-capturing component.

10. The endoscope tip according to claim 7, wherein the light-guiding structure comprises a third portion, the third portion is connected to another end of the first portion opposite to the second portion and extends in a varying cross section along the lengthwise direction, and the third portion is between the first portion and the light-emitting component.

11. The endoscope tip according to claim 7, wherein the light-guiding structure has a flat light-incident surface, a normal direction of the flat light-incident surface and the lengthwise direction form an acute angle, the light-emitting component has a light-emitting surface parallel to the flat light-incident surface.

12. The endoscope tip according to claim 11, wherein the flat light-incident surface and the light-emitting surface are bonded with an optically clear adhesive.

13. The endoscope tip according to claim 7, wherein the light-guiding structure has a flat light-incident surface, the light-emitting component has a light-emitting surface, and the light-emitting surface and the flat light-incident surface are non-parallel.

14. The endoscope tip according to claim 7, wherein the first and second portions are formed as a single part of the same material.

15. The endoscope tip according to claim 7, further comprising a tip housing, wherein the tip housing accommodates the circuit board, the image-capturing component, the light-emitting component, and the light-guiding structure.

16. The endoscope tip according to claim 7, wherein a direction of the light-emitting component is adjustable.

17. A method for manufacturing a light-guiding structure used in an endoscope, the method comprising the following steps:
(a) providing a light-transmissive structure, the light-transmissive structure extending in a single cross section along a lengthwise direction and having a first end and a second end opposite to the first end in the lengthwise direction; and
(b) shaping a portion of the light-transmissive structure with the second end so that the shaped portion extends in a varying cross section along the lengthwise direction, so as to complete the light-guiding structure.

18. The method according to claim 17, wherein the step (b) comprises using a mold to shaping the portion of the light-transmissive structure.

19. The method according to claim 18, wherein the step (b) comprises heating the mold before the mold shapes the portion of the light-transmissive structure.

20. The method according to claim 17, wherein the step (b) comprises shaping the portion of the light-transmissive structure with the second end so that the shaped portion has a component accommodating space.

21. The method according to claim 20, wherein the step (b) comprises shaping the portion of the light-transmissive structure with the second end so that the shaped portion has an annular segment light-emitting surface at a side of the component accommodating space.

22. The method according to claim 17, further comprising the following step:
(c) shaping the first end of the light-transmissive structure to form a flat light-incident surface, of which a normal direction and the lengthwise direction forms an acute angle.

23. A method for manufacturing a light-guiding structure used in an endoscope, the method comprising the following steps:
(a) providing a light-transmissive structure, the light-transmissive structure extending in a single cross section along a lengthwise direction and having a first end and a second end opposite to the first end in the lengthwise direction;
(b) providing a mold with a cavity;
(c) disposing the light-transmissive structure in the mold so that the second end is exposed in the cavity;
(d) filling the cavity with a material into the cavity; and
(e) solidifying the material in the cavity so that the solidified material is directly bonded to the second end and extends in a varying cross section along the lengthwise direction, so as to complete the light-guiding structure.

24. The method according to claim 23, wherein the mold is light-transmissive, the material is photopolymer, and the step (d) comprises curing the material in the cavity with a light.

25. The method according to claim 23, further comprising the following step:
(f) shaping the first end of the light-transmissive structure to form a flat light-incident surface, of which a normal direction and the lengthwise direction forms an acute angle.
